(19) United States
(12) Patent Application Publication
(10) Pub. No.: US 2013/0224788 A1
(43) Pub. Date: Aug. 29, 2013

(54) METHOD FOR DETECTING MICROORGANISMS WITH A SPECIMEN

(71) Applicant: NANECTIS BIOTECHNOLOGIES SA, (US)

(72) Inventors: Luc MONTAGNIER, Le Plessis Robinson (FR); Jamal AISSA, Chatillon (FR)

(73) Assignee: NANECTIS BIOTECHNOLOGIES SA, Jouy en Josas (FR)

(21) Appl. No.: 13/785,718

(22) Filed: Mar. 5, 2013

Related U.S. Application Data
(63) Continuation of application No. 12/305,417, filed on Nov. 30, 2009.

(30) Foreign Application Priority Data
Jun. 22, 2006 (FR) 0605599

(51) Int. Cl. C12Q 1/04

(52) U.S. Cl. CPC C12Q 1/04
USPC 435/34; 435/288.7

(57) ABSTRACT

A process for preparing reagents for a microorganism detection test comprising: a) centrifuging medium containing a microorganism; b) filtrating the supernatant from (a); c) preparing diluted samples of the filtrate from (b); d) treating the diluted samples from (c) with an E/M field; e) detecting signals emitted from (d) using a solenoid; f) selecting samples from (e) whose signal amplitude is ≧1.5 times greater than background noise emitted by water or that present a frequency displacement towards higher values; g) placing the diluted samples from (f) into an enclosures protecting against external electromagnetic fields; h) splitting a diluted sample from (g), volume by volume, into two tubes, T1 and T2, where tube T1 is protected from external electromagnetic field interferences, and reference tube T2 is also placed in a protective enclosure and subjected subsequently to the presence or contact of a sample suspected of containing a specific microorganism.

### CROSS-REFERENCE TO RELATED APPLICATIONS

This application is a Continuation of U.S. application Ser. No. 12/305,417, filed Nov. 30, 2009 that is a U.S. National Stage entry of International Application No. PCT/FR2007/001042, filed on Jun. 22, 2007, which claims the priority of French Application No. 0605599, filed on Jun. 22, 2006, in France. This application incorporates by reference the full disclosures of all of the above-mentioned applications.

This invention has for object to reveal latent infections in humans and animals, by showing inhibition, through the examinee, of electromagnetic signals generated by a microorganism.

From the works by Dr. Jacques BENVENISTE and from patent application WO 00/17637, it has been known how to record and digitalize, after analog-to-digital conversion using a computer sound board, an electrical signal characteristic of a molecule possessing a biological activity.

Also known in prior art (WO 09417406) is a process and a device used to transmit biological activity from a first matter, so-called carrier, to a second matter, so-called target, the latter exempted of any traces from said carrier and physically separate from it, and the target not presenting initially the aforementioned biological activity. The method consists in (i) exposing the matter carrying the biological activity of interest to an electrical or electromagnetic signal sensor, (ii) amplifying said electromagnetic or electrical signals characteristic of the emitted biological activity feature, then (iii) exposing the target matter to an emitter of electrical or electromagnetic signals, said emitter being connected to aforesaid sensor through a transmission and amplification circuit, in order to transmit the signal characteristic of biological activity to said target.

In a previous French patent application 05/12686 filed on Dec. 14, 2005, not yet issued to this day, the inventor of this invention was describing a process for characterizing biochemical elements presenting a biological activity, microorganisms in this case, by analyzing low frequency electromagnetic signals, said process bringing improvements to prior art techniques. Said process also relates to biological analysis consisting in recording the electromagnetic or electrical “signatures” corresponding to known biochemical elements, and to compare such pre-recorded “signatures” to that of a biochemical element to be characterized. Said process implicates filtration and dilution steps in order to eliminate microorganisms and cells present within the original sample, the highest dilutions generating the most electrical or electromagnetic signals whereas the least diluted samples don't provide, most of the time, any electrical or electromagnetic signals. The inventor also showed that microorganisms of different nature, such as bacteria and viruses, produce “nanostructures” that persist in aqueous solutions, and that these very “nanostructures” are emitting electromagnetic signals. Said “nanostructures” behaves like polymers of a size less than 0.02 .mu.m for viruses, and less than 0.1/.mu.m for classic size bacteria, and present a density ranging from 1.12 and 1.30 g/ml.

The process described in this application is based on the astonishing observation that in absence of physical contact, the mere vicinity of a closed tube containing a sample of a bacterial or viral filtrate, little diluted and negative with regard to electrical or electromagnetic emitting signals, inhibits the signals produced by a more diluted sample of the same filtrate, initially positive with regard to electrical or electromagnetic signal emission. In this application, such inhibition will be indistinctly called “inhibitory effect” or “negativing effect”. In the same way, in this application, to “inhibit” and “negativate” will be used indistinctly and have a similar meaning. This observation led the inventor to search for the same inhibitory phenomenon from an infected human being. It has been observed, in a patient suffering from an auto-immune microvascularitis of infectious origin, that the diluted samples of his plasma had an inhibitory effect on dilute filtrates of E. coli emitting electromagnetic signals (hereafter EMS), suggesting that the patient was suffering from a chronic infection by this or a related germ. It was also shown that the patient suffering from microvascularitis, as mentioned in the previous example, himself inhibits the EMS emitted by his filtered and diluted plasma, and also inhibits the EMS emitted by a filtered and diluted sample of E. coli culture present in a closed tube. In this case, a 5 minutes contact of a positive dilution in the patient's hand, or 10 minutes at a distance of up to 50 cm, are sufficient to observe said inhibitory effect.

Said inhibitory power thus involves both the emitting structures from one own plasma, and those of a specific bacterial germ, which could thus be used as a universal identification system.

The invention may therefore enable to determine a bacterial or viral origin in illnesses where such germs have not been identified.

A first object of the invention concerns a method for preparing reagents to be used in a test for detecting a microorganism and notably an infection in humans or animals. According to its most general acception, the method includes the following steps:
a) Centrifuging a biological or artificial liquid medium containing a selected specific microorganism; b) Filtrating the supernatant obtained at step (a); c) Preparing a series of diluted samples corresponding to increasing dilutions of the filtrate obtained in step (b), down to a filtrate dilution factor of at least 10.sup.-15; d) Submitting the diluted samples obtained in step (c) to an electrical, magnetic and/or electromagnetic exciting field; e)

Analyzing the electrical signals detected using a solenoid and recording digitally aforesaid electrical signal, after analog/digital conversion of aforesaid signal; f) Selecting diluted samples from which the characteristic electrical signals were obtained in (e), by characteristic signals one means signals whose amplitude is at least 1.5 times greater than background noise emitted by water, and/or presenting a frequency displacement towards higher values; g) Introducing the diluted samples selected in step (0 in protective enclosures, which protect said dilutions from very low frequency external electromagnetic fields; h) Distributing one of aforesaid diluted samples from step (g), volume by volume, in two tubes, T1 and T2, with T1 remaining in a protective enclosure protecting said diluted samples from external electromagnetic field interferences, said tube T1 acting as a reference solution, while tube T2, also placed in a protective enclosure, is subsequently being subjected to the presence or contact of a sample suspected of containing said selected specific microorganism.

By “a sample to be tested for presence or absence of aforesaid selected specific microorganism” one means: (i) a human or animal individual suspected to be infected by aforesaid selected specific microorganism, or (ii) a biological specimen or a biological or artificial fluid suspected of containing said selected specific microorganism, or (iii) a food component, a cosmetic, or a pharmaceutical composition susceptible to contain said selected specific microorganism.

By biological fluids, one means any human or animal fluid, e.g. blood, urine, various secretions. By artificial fluid, one means any reconstituted fluid for growing microorganisms, e.g. various culture media for bacteria, yeasts, and molds, and culture media for cells infected by a virus.

Another object of the invention concerns a system for detecting a microorganism within a sample. This system includes:
a) A tube T1 containing a reference sample emitting characteristic electrical signals, by characteristic signals one means signals whose amplitude is at least 1.5 times greater than background noise emitted by water, and/or presenting a frequency displacement towards higher values; b) A tube T2 containing a sample emitting characteristic electromagnetic signal, said sample being identical to that contained in tube T1; c) A protective enclosure for protecting tubes T1 and T2 against very low frequency external electromagnetic fields; d) A tube T3 containing a control solution not presenting electromagnetic signal emission; e) An equipment for receiving electromagnetic signals.

During detection, tube T2 will be subjected to the presence or contact of sample X to be tested for presence or absence of a selected specific microorganism.

Another object of the invention concerns a method for detecting a microorganism within a sample, characterized in that said method consists of the following steps:
a) A sample X, for which the presence of a suspected microorganism, e.g. E. coli, is to be established, is exposed to a sample as obtained after step (f) of the process according to one of claims 1 to 3, said sample obtained after step (f) being a dilution of a culture or biological medium filtrate containing said microorganism suspected to be present in sample X; b) Comparing the electromagnetic signal emitted by sample X exposed to said sample obtained after step (f), obtained in step (a), with the electromagnetic signal emitted by an aliquot of the same sample obtained after step (f) and not submitted to sample X.

By “a sample X”, one means (i) a human individual or animal suspected of being infected by aforesaid selected specific microorganism, or (ii) a biological specimen, or a biological or artificial fluid, suspected to contain said selected specific microorganism, or (iii) a food component, cosmetic, or pharmaceutical composition susceptible to contain said selected specific microorganism.

The methods according to the invention enable (i) to prepare reagents intended for a test to detect microorganisms implicated in chronic illnesses, and/or intended to detect systemic latent infections under circumstances where a quick and non invasive response is required, as it is in the case of e.g. avian flu virus detection, (ii) the identification of an infection in humans or animals.

Once the responsible microorganism identified, it is possible to confirm the presence of that germ using supersensitive PCR with specific oligonucleotidic promoters from such microorganism.

The invention shall be better understood by reading the following description, presenting in a non restrictive way examples of process embodiment according to the invention.

The figures in annex correspond to non restrictive examples of embodiment.

### Example 1

A Lightly Dilute Bacterial Culture, not Emitting Electromagnetic Signals, “Negates” the Electromagnetic Signals Emitted by a Strong Dilution from the Same Culture

### 1) Sample Preparation

An Escherichia coli (E. coli) bacteria culture in LB (Luria broth) medium is centrifuged at 8000 rpm for 15 minutes in order to eliminate the cells. The bacterial supernatant is then filtered on a 0.45 .mu.m porosity PEVD Millipore filter, and the filtrate is then again filtered on a 0.1 .mu.m porosity Millipore filter.

From the resulting E. coli culture filtrate, which is completely sterile, one prepares a series of samples by diluting the filtrate from 10 to 10 into water down to 10.sup.-15 for injectable preparation. The successive dilutions are strongly agitated with a vortex for 15 seconds between each dilution.

The diluted samples are distributed in 1.5 ml Eppendorf conic plastic tubes. The fluid volume is in general of 1 milliliter.

### 2) Selection of Diluted Samples Generating Electromagnetic Signals.

Each dilute sample is tested for emission of low frequency electromagnetic signals.

The procedure for detecting EMS includes a step aimed at transforming the electromagnetic field from various diluted samples into one signal, namely an electrical signal, using a solenoid for capturing said electromagnetic field.

The transformation of the electromagnetic field coming from the diluted sample analyzed into an electrical signal is done as follows:
(i) Submitting the dilute sample being checked to an electrical, magnetic and/or electromagnetic exciting field; (ii) Analyzing the electrical signals detected using a solenoid and digitally recording aforesaid electrical signal after analog/digital conversion of said signal; (iii) Selecting the diluted samples generating characteristic electrical signals, by ‘characteristic’ one means signals whose amplitude is at least 1.5 times greater than background noise signals emitted by water and/or presenting a frequency displacement towards higher values, and placing them in Mumetal.®. protective enclosures for protecting said diluted samples against external electromagnetic field interferences.

Signal detection is carried out using the equipment schematically represented in FIG. 1. The equipment consists of a solenoid reading cell (1) sensitive from 0 to 20000 hertz, placed on a table made of insulating material. Said solenoid used in step (ii) includes a winding comprising a soft iron core. Said winding has an impedance of 300 ohms, an inside diameter of 6 mm, an outside diameter of 16 mm, and a length of 6 mm. The magnetic soft iron core is placed in contact with the external walls of the tube containing the dilution to be analyzed.

The diluted samples to be read are distributed in 1.5 ml Eppendorf (trade mark) conic plastic tubes (2). The fluid volume is in general of 1 milliliter.

Characteristic electrical signal acquisition is performed for a preset duration, i.e. ranging from 1 to 60s. In this example, each sample is read twice successively for 6 seconds.

The electrical signals delivered by the solenoid are amplified and converted into analog-digital signals using a signal acquisition board (sound card) (4) including a computer-built-in analog-to-digital converter (3). Said analog-to-digital converter has twice the sampling rate of the maximal frequency that one wants to digitalize, e.g. 44 kHz.

The digital file corresponding to said converted electrical signal is saved on a mass storage, e.g. as a WAV format audio file.

For processing the characteristic electrical signal, one uses e.g. Matlabs and SigViews (trademarks) software. The recorded digital file may possibly undergo digital processing, i.e. digital amplification for calibrating the signal level, filtering for eliminating undesired frequencies, calculating spectral power distribution (SPD), then such spectral power is truncated, e.g. only keeping frequency bands from 140 Hz to 20 kHz (Matlab), or is transformed in frequency components by Fourier transform (SigView).

### 3) Evaluating the Inhibitory Activity of a Non-Emitting Low Dilution on the Emission of Electromagnetic Signals Generated by an Active Dilution.

The diluted samples presenting characteristic electrical signals are samples diluted to 10.sup.-8, 10.sup.-9, 10.sup.-10. The 10.sup.-2 to l0.sup.-6 dilutions are negative (FIG. 2).

A closed tube containing a 10.sup.-3 dilution aliquot of E. coli is placed side by side with a closed tube containing a 10.sup.-8 diluted sample aliquot of E. coli, in an enclosure surrounded by a Mumetal.®. magnetic shield, and left 24 hours at room temperature. In parallel, a control series is realized. This control series consists of one tube containing a 10.sup.-3 diluted sample aliquot of E. coli, and of another containing a 10.sup.-8 diluted sample aliquot of E. coli that is processed in the same way, but in separate Mumetal.®. enclosures distant from one another. The placement in a Mumetal.®. enclosure eliminates very low active frequencies (5 to 100 Hertz) but not higher frequencies that could come from ambient electromagnetic noise.

After 24 hours, the tubes containing the diluted samples are again analyzed as describes above, revealing that the tube containing a 10.sup.-8 diluted sample aliquot and coupled to the tube containing a 10.sup.-3 diluted sample aliquot, no longer emits any electromagnetic signals, or much weaker ones. On the other hand, the control series tubes remained identical; the tube containing a 10.sup.-8 diluted sample aliquot protected from contact with the tube containing a 10.sup.-3 diluted sample aliquot remained positive for electromagnetic signal emission.

An important particularity of the invention is that the observed negating effect is specific, i.e. the lightly diluted, non-emitting sample and the greatly diluted electromagnetic signal-emitting sample must come from the same microorganism species.

Thus, the diluted E. coli-emitting samples are only “negated” by a weakly diluted non-emitting E. coli sample, but not by a lightly diluted non-emitting Streptococcus or Staphylococcus sample. Similarly, a diluted emitting Staphylococcus sample is only “negated” by a lightly diluted non-emitting sample of Staphylococcus and not by a lightly diluted non-emitting sample of Streptococcus or E. coli.

### Example 2

Quick and Non-Invasive Method for Detecting Infections in Humans and Animals

### 1) Preparations of Biological and Artificial Fluid Samples Containing Microorganisms.

A blood sample, collected with anticoagulant, preferably heparin, from a patient suffering from a neurological pathology consecutive to a bacterial infection, and an Escherichia coli (E. coli) bacteria K1 culture in suspension in LB (Luria broth) medium are centrifuged in order to eliminate the cells. The bacterial supernatant and/or the plasma collected are then diluted to 10.sup.-2 in RPMI medium. The solutions are filtered on 0.45.mu. Millipore PEVD filter, then the filtrate is again filtered on 0.02 .mu.m Whatman or 0.1 .mu.m Millipore filter.

From the plasma filtrates of infected individual and from the E. coli K1 culture, one prepares a series of diluted samples corresponding to increasing dilution levels, up to 10.sup.-15, in 10 to 10 dilutions in water for injectable preparation under laminar flow hood. The successive dilutions are strongly agitated with a vortex for 15 seconds between each dilution.

The diluted samples are then distributed in 1.5 ml conic Eppendorf plastic tubes. The fluid volume is in general of 1 milliliter.

### 2) Selection of Diluted Samples Generating Electromagnetic Signals.

The selection of the diluted samples emitting characteristic signals, signals whose amplitude is at least 1.5 times greater than the background noise signals and/or are of a frequency higher than background noise, is realized identically to what is described above in example 1, chapter 2. The method described as well as the material are identical to what is described above. Thus, the method includes a step for transforming the electromagnetic field from different dilutions into a signal, namely an electrical signal, by means of a solenoid capturing said electromagnetic field.

The transformation of the electromagnetic field from the analyzed dilution into an electrical signal is done by: (i) Submitting the diluted sample being checked to an electrical, magnetic and/or electromagnetic exciting field; (ii) Analyzing the electrical signals detected using a solenoid, and digitally recording said electrical signal after analog/digital conversion of aforesaid signal; (iii) Selecting the diluted samples presenting characteristic electrical signals, by ‘characteristic’ one means signals whose amplitude is at least 1.5 times greater than background noise signals emitted by water, and/or presenting a frequency displacement towards higher values, and placing them in protective enclosures for protecting said diluted samples against external electromagnetic field interferences.

### 3) Evaluating an Infected Individual's Inhibitory Activity on the Electromagnetic Signal Emission Generated by a Microorganism.

The diluted samples selected at the previous step (item (iii)), from the plasma filtrate of the infected individual, from E. coli culture filtrate, i.e. the dilutions of filtered sample presenting a characteristic electrical signal, are distributed in Eppendorfs plastic tubes, at a rate of 1 ml per tube, and stored at +4.degree. C. The diluted EMS emitting samples distributed in aliquots are protected from external influences by being placed in an enclosure protected from electromagnetic fields. Preferably, the enclosure is surrounded with a magnetic shield made of Mumetal.®., isolating the enclosure from very low frequency parasitic fields coming from the surroundings.

One of the diluted EMS emitting samples from the plasma filtrate of the infected individual, from E. coli culture filtrate, is distributed volume to volume in two tubes, T1 and T2, with T1 remaining in a protective enclosure protecting said diluted samples from external electromagnetic field interferences, that tube will act as reference solution; tube T2 will be subsequently subjected to the patient and is also placed in a protective enclosure.

Said protective enclosure being preferably surrounded with a Mumetal.®. shield.

FIG. 2 represents schematically the steps to take when searching for the inhibitory effect. The search of the inhibitory effect is realized as follows: a) Tube T1, containing the reference solution, remains in an enclosure (3) surrounded by a Mumetal.®. magnetic shield, said tube T1 is thus protected from potential changes of the individual to be examined (4), whereas tube T2 is submitted to the influence of the infected individual to be examined (4) whose plasma present in tubes T1 and T2 comes from, said individual holds T2 in his/her hand (5) for a set period of time, e.g. 5 minutes; b) Tube T2 is placed in an electromagnetic signal reception equipment, preferably a reading solenoid cell as described previously in chapter 2 of this example; c) Electrical signals are then amplified, processed, converted into analog-digital signals as previously described in chapter 2; d) Said analog-digital signals are possibly decomposed in harmonics by Fourrier transform.

The signals corresponding to tube T1 and those corresponding to tube T2, as well as the signals corresponding to water containing tube T3 (background noises) are compared.

The following figures represent the results obtained in the case where the active dilution comes from the examined infected individual plasma:

### BRIEF DESCRIPTION OF THE DRAWINGS

FIG. 1 depicts signal detection equipment. (1) solenoid reading cell. (2) Plastic tubes. (3) Computer-built-in analog-to-digital converter. (4) Signal acquisition board.

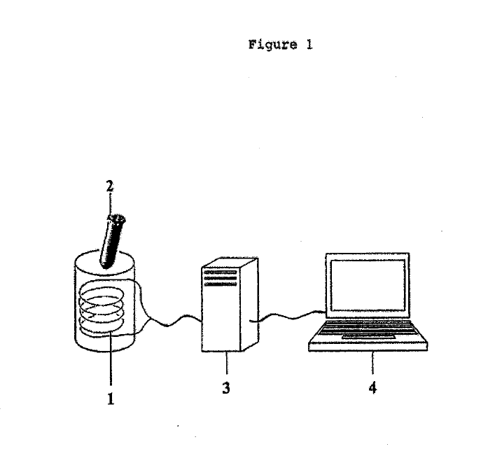

FIG. 2. General diagram of the capture device. (1) Tube T1. (2) Tube T2. (3) Enclosure surrounded by mu metal magnetic shield. (4) Infected individual providing blood plasma. (5) Hand of infected individual.

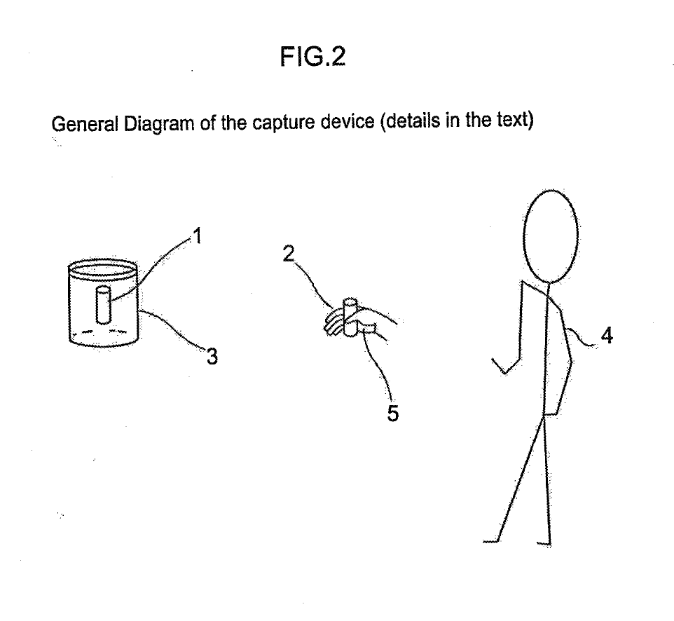

FIG. 3 represents a histogram in three dimensions (Matlab) of the electrical signals detected by the solenoid with tube T3 present (background noises);

FIG. 4 represents a three dimension histogram of the frequency spectrum detected by the solenoid with tube 1 present;

FIG. 5 represents a three dimension histogram of the frequency spectrum detected by the solenoid with tube 2 present;

FIG. 6 represents a Fourier analysis (SigView) of the same background noise (the harmonics of the non-filtered current of the power supply);

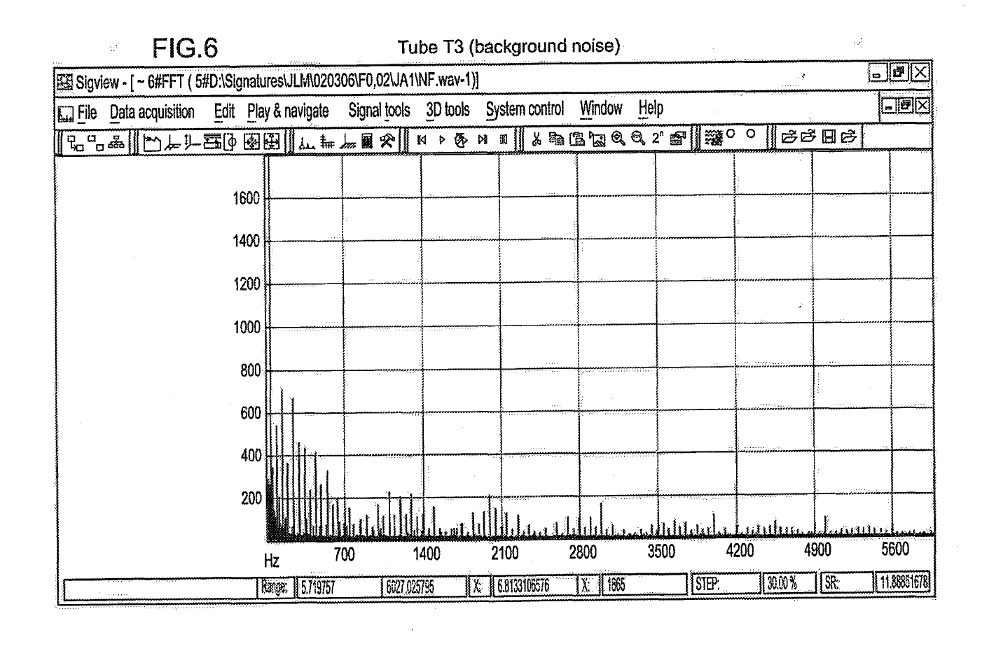

FIG. 7 represents a Fourier analysis of the signal detected by the solenoid with tube 1 present;

FIG. 8 represents a Fourier analysis of the frequency spectrum detected by the solenoid with tube 2 present, handled by the individual to be examined.

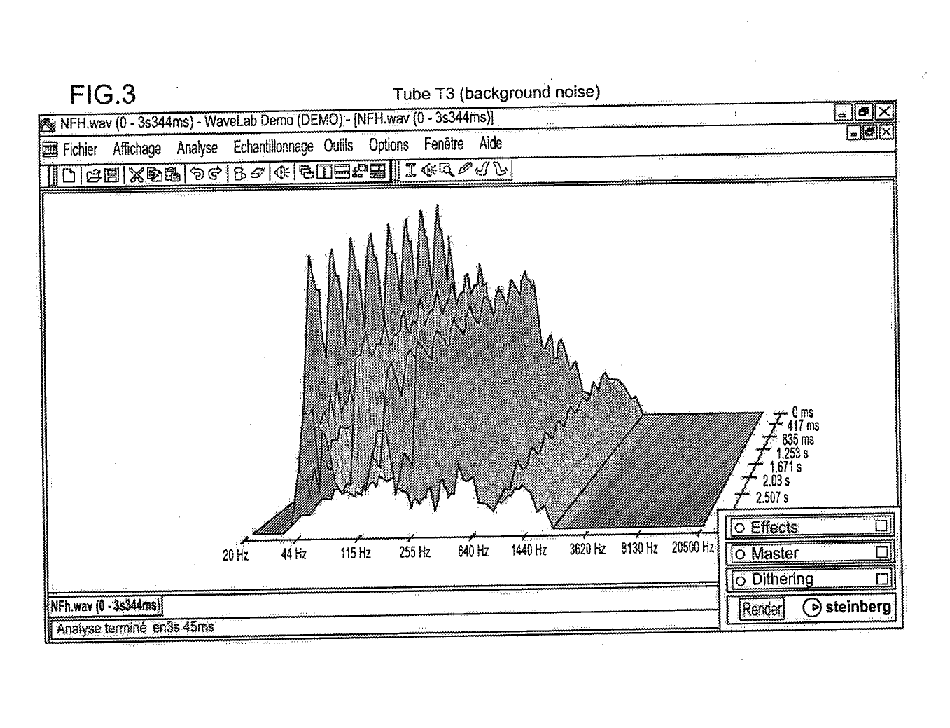

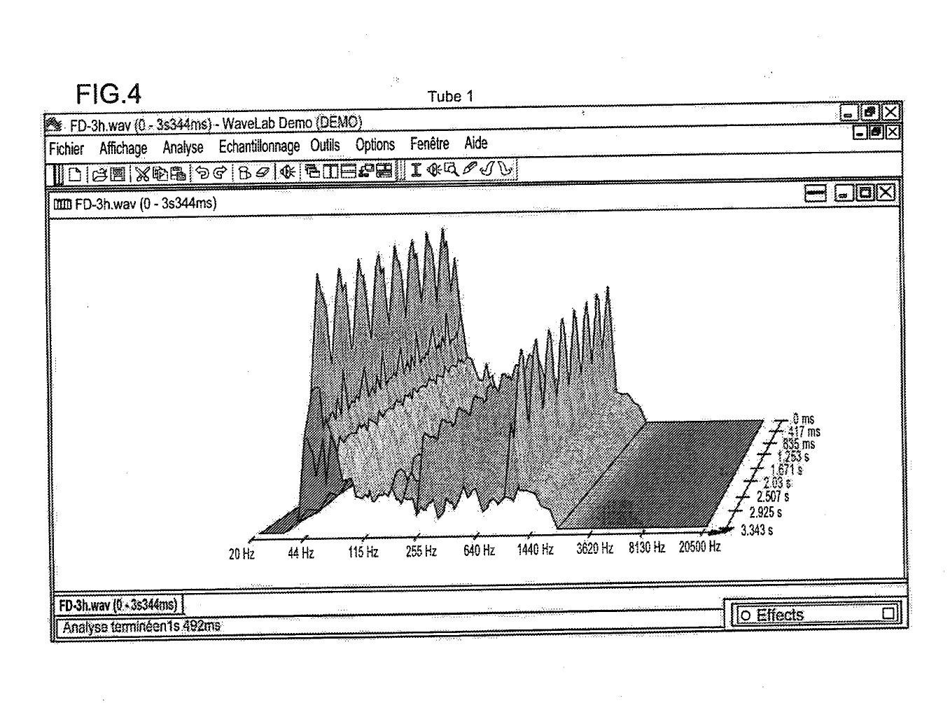

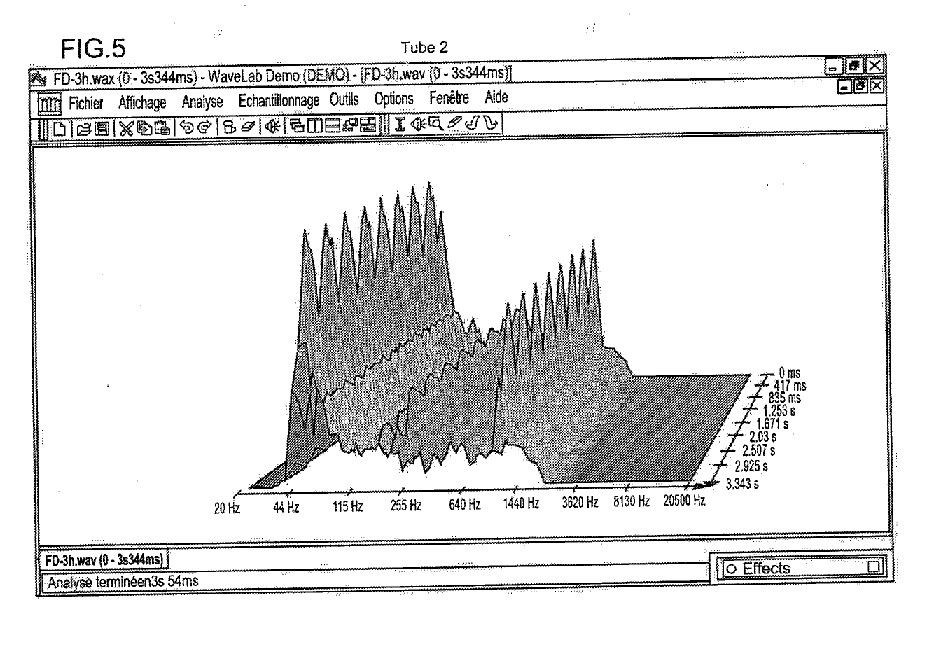

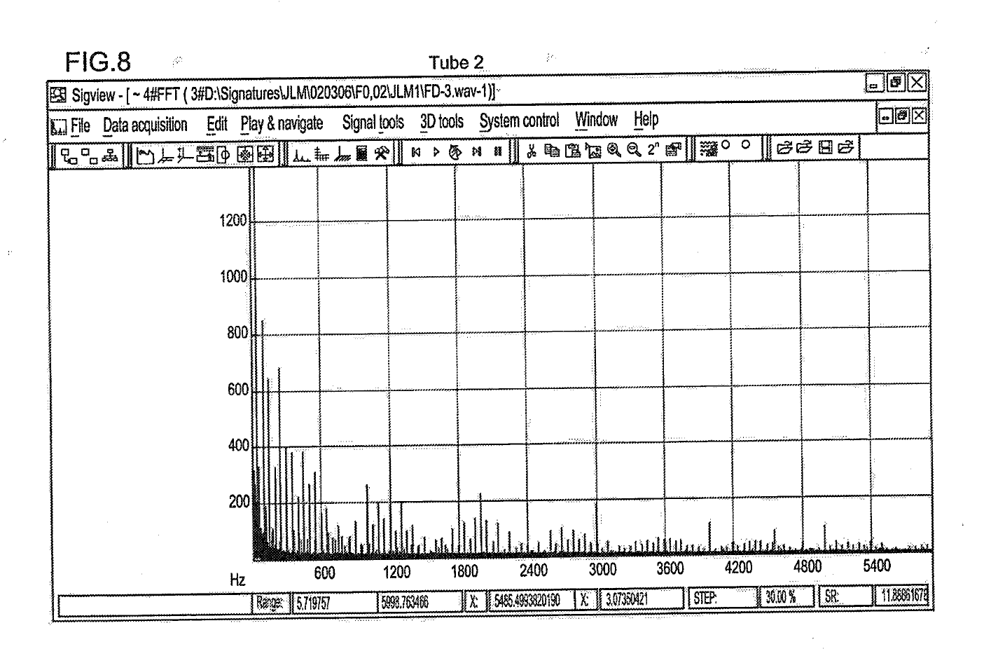

The analysis by 3 dimensions histogram, respectively for background noise (FIG. 3) and for the signal obtained with tube T1 present and containing the EMS emitting reference solution (FIG. 4), shows a displacement towards higher frequencies. On the other hand, when analyzing tube T2 containing the solution submitted to the influence of the individual to be examined (FIG. 5), no displacement toward higher frequencies is noted; the 3D histogram representing the signals of tube T2 is analogous to that obtained for background noise.

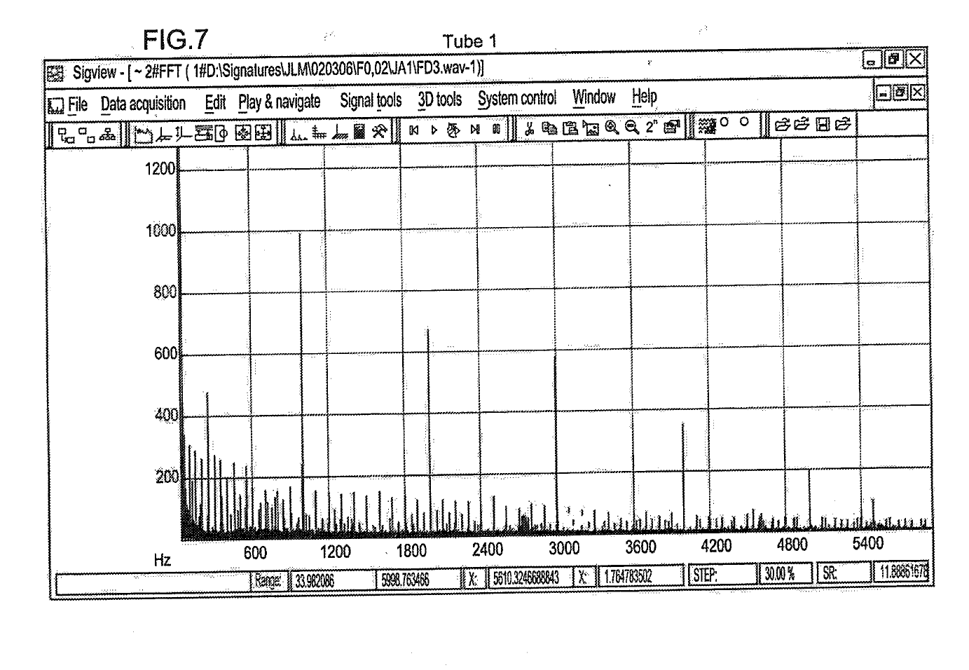

Fourier analysis of the positive frequencies generated by tube 1 (FIG. 7) revealed peaks at various frequencies. By decreasing order of signal intensity, the following frequencies presented signals: 1000, 2000, 3000, 4100, 5100 and 5500. On the other hand, Fourier analysis of tube T2 reveals results analogous to those obtained by background noise analysis: no significant peak was observed for background noise or for tube T2.

In conclusion, these analyses enable to deduct that the individual examined has a capacity for inhibiting electromagnetic signals emitted by a dilution of his/her own plasma.

Analogous results were obtained with the reference solution, derived from K1 E. coli.

Therefore, this inhibitory capacity concerns not only his/her own plasma but also E. coli emitting structures, suggesting that the individual is infected by an agent producing nanostructures close to those of E. coli.

1-11. (canceled)

12. A process for preparing a reagent for use in a microorganism detection test, comprising:
a) centrifuging a biological or artificial liquid medium containing a microorganism, thereby resulting in a supernatant and a pellet;
b) filtering the supernatant obtained in step (a), thereby resulting in a filtrate;
c) preparing a series of tenfold dilutions of the filtrate obtained in step (b), down to a filtrate dilution of a factor of 10⁻¹⁵, thereby resulting in diluted samples;
d) submitting said diluted samples obtained in step (c) to an electrical, magnetic, and/or electromagnetic exciting field, thereby resulting in electrical signals;
e) detecting and analyzing the electrical signals of step (d) using a solenoid and converting the electrical signals from analog form to digital form, and digitally recording said electrical signals;
f) selecting diluted samples from which the electrical signals detected and analyzed in step (e) have amplitudes that are at least 1.5 times greater than background noise signals emitted by water and/or are of a frequency higher that background noise signals emitted by water;
g) placing the diluted samples selected in step (f) into protective enclosures, which protect said dilutions against external electromagnetic fields;
h) distributing one of the aforesaid diluted samples from step (g) into two tubes, T1 and T2, that are provided in a protective enclosure which protects said diluted samples from external electromagnetic field interferences, wherein the diluted sample provided in tube T1 is a reference solution, and the diluted sample provided in tube T2 is a solution used to test a sample suspected of containing said microorganism, thereby obtaining reactants for a microorganism detection test.

13. The process according to claim 12, wherein the biological liquid medium is a liquid of human or animal origin.

14. The process according to claim 12, wherein the artificial liquid medium is a microorganism culture medium.

15. A process for determining the presence of a microorganism within a sample, wherein said process consists of the following steps:
a) providing a sample x, in which the presence of a microorganism is suspected;
b) exposing said sample x to a sample obtained after step (f) of the process according to claim 1, said sample obtained after step (f) being a dilution from a culture or biological medium filtrate that contains said microorganism suspected to be present in sample x;
c) comparing an electromagnetic signal emitted by the sample as defined in step (f) of claim 1 after having been exposed to sample x, with an electromagnetic signal emitted by an aliquot of the same sample as obtained after step (f) of the process of claim 1 which was not exposed to the sample x, wherein an inhibition of the electromagnetic signal emitted by said sample after having been exposed to sample x indicates the presence of a microorganism in the sample x.

16. The process according to claim 15, wherein said sample x is a human being or an animal.

17. The process according to claim 15, wherein said sample x is a biological fluid or an artificial fluid.

18. The process according to claim 15, wherein said sample X is a food, cosmetic, or pharmaceutical composition.

19. A system for detecting a microorganism within a liquid sample comprising:
a) a tube T1 prepared according to the process of claim 1, containing a liquid reference sample emitting characteristic electromagnetic signals of a microorganism, wherein said characteristic electromagnetic signals of said microorganism have an amplitude that is at least 1.5 times greater than background noise signals emitted by water, and/or are of a frequency higher than background noise signals emitted by water;
b) a tube T2 prepared according to the process of claim 1, containing a liquid sample emitting characteristic electromagnetic signals of a microorganism, said sample being identical to that contained in tube T1;
c) a protective enclosure protecting tubes T1 and T2 against external electromagnetic fields;
d) a tube T3 containing a negative control solution without electromagnetic signal emission; and
e) equipment for receiving electromagnetic signals.

20. The system according to claim 19, wherein the electromagnetic signal receiving equipment (e) comprises:
a reading solenoid cell;
a computer provided with a signal acquisition board, said computer including at least one software for processing the signals.

21. System according to claim 20, wherein the reading solenoid cell is sensitive from 0 to 20000 hertz, includes a winding with soft iron core, said winding having an impedance of 300 ohms, an inside diameter of 6 mm, an outside diameter of 16 mm, a length of 6 mm.

22. The system according to claim 19, wherein a negative control solution T3 is used to dilute the sample ending in tubes T1 and T2.

23. A process for preparing a reagent that can identify a microorganism in a microorganism detection test, comprising:
filtering a liquid biological sample which contains an isolated microorganism to produce a filtrate;
serially diluting the filtrate with strong agitation between each serial dilution, thereby producing a series of serially-diluted samples;
exposing the serially-diluted samples to an exciting electrical, magnetic, and/or electromagnetic field;
selecting a serially-diluted sample emitting electromagnetic signals (EMS) having an amplitude that is at least 1.5 times greater than background noise signals emitted by water and/or having a frequency higher that background noise signals emitted by water, thereby identifying a reagent that can be used to detect the microorganism; and
storing the reagent emitting EMS in an enclosure which protects it from external electromagnetic field interferences.

24. The process of claim 23, further comprising distributing two identical samples of the reagent emitting EMS into two tubes T1 and T2.

25. The process of claim 23, further comprising centrifuging the biological sample to remove cells prior to filtration.

26. The process of claim 23, wherein the filtrate is recovered after filtration through a 0.45 μm filter and then through a 0.1 μm filter.

27. The process of claim 23, wherein ten-fold serial dilutions are made in water down to a dilution factor of at least 10⁻¹⁵.

28. The process of claim 23, wherein selecting a serially-diluted sample emitting EMS comprises detecting EMS using a solenoid sensitive from 0 to 20,000 Hz, converting the detected EMS from analog form to digital form, and digitally recording the EMS.

29. A reagent produced by the process of claim 23.

30. A reagent that emits electromagnetic signals (EMS) having an amplitude that is at least 1.5 times greater than background noise signals emitted by water and/or having a frequency higher that background noise signals emitted by water;
wherein said EMS are characteristic of a microorganism and are inhibited by bringing a closed sample of said reagent into contact with a biological sample containing said microorganism for 5 minutes or within a distance of 50 cm to said biological sample for 10 minutes.

31. A kit comprising two identical samples, T1 and T2, of the reagent emitting EMS produced by the process of claim 23 inside of a protective enclosure, and optionally a control sample, T3, containing a control solution not presenting EMS.

32. A method for detecting the presence of a microorganism in a biological sample to be tested comprising:
keeping a reagent sample T1 inside of a protective enclosure and not exposing it to the biological sample to be tested;
contacting at a distance up to 50 cm a closed reagent sample T2 made by the process of claim 23 with the biological sample to be tested for the microorganism used to produce reagent samples T1 and T2 by the process of claim 23;
comparing electromagnetic signals (EMS) emitted by reagent sample T1 to EMS emitted by sample T2 after T2 has been exposed to the biological sample;
determining the presence of the microorganism in the biological sample when the EMS emitted by sample T2 is less than that emitted by sample T1.
